(19) United States
(12) Patent Application Publication
(10) Pub. No.: US 2022/0249766 A1
(43) Pub. Date: Aug. 11, 2022

(54) PEN ASSEMBLY

(71) Applicant: Becton, Dickinson and Company, Franklin Lakes, NJ (US)

(72) Inventors: Amit Limaye, Wayne, NJ (US); Dipumon Ayyanchira Mani, Kerala (IN); Praveesh Karattu Meethal, Kerala (IN); Sajayesh Vijayachandran, Kerala (IN)

(73) Assignee: Becton, Dickinson and Company, Franklin Lakes, NJ (US)

(21) Appl. No.: 17/615,585

(22) PCT Filed: Apr. 26, 2019
(86) PCT No.: PCT/US19/29329

§ 371 Date: Dec. 1, 2021

(51) Int. Cl. A61M 5/00; A61M 5/42; A61M 5/50; A61M 5/32

(52) U.S. Cl. CPC A61M 5/002; A61M 5/427; A61M 2005/004; A61M 5/3202; A61M 5/3234; A61M 5/5086

(57) ABSTRACT

A needle magazine (30) comprising a plurality of cavities (38) each carrying a needle (74) fixed to a needle post (70), a first seal (46) disposed at a proximal end of each of the plurality of cavities (38), and a second seal (48) disposed at a distal end of each of the plurality of cavities (38), wherein in a first position of the needle post (70), the needle (74) is enclosed between the first and second seals (46, 48), in a second position of the needle post (70), the first seal (46) is pierced and the needle (74) pierces and extends through the second seal (48) for medication delivery, and in a third position of the needle post (70), the needle (74) no longer extends through the second seal (48) and is ready for disposal.

### FIELD OF THE INVENTION

Various exemplary embodiments of the invention relate to needle storage. use and disposal for medication delivery pens.

### BACKGROUND OF THE INVENTION

Medication delivery pens are typically used to inject medication into a patient. A person who must periodically self-inject doses of medication will typically carry a medication delivery pen, several single-use pen needles, and several cleaning swabs. A medication delivery pen is designed for safety and sterility. However, inefficiencies and inconveniences can arise.

### SUMMARY OF THE INVENTION

It is an aspect of the present invention to provide a needle storage container that stores two or more needle magazines, each carrying a plurality of needles. Each of the plurality of needles is configured to engage a medication delivery pen for medication delivery. Specifically, the medication delivery pen engages one of the plurality of needles and provides medication delivery while the needle is in the needle magazine. The combination of the container and the magazine provides advantages in easy organization, accurate subcutaneous injection, allows for needle disposal after use and storage before use in the same magazine, and improves usage with an optimized workflow to reduce setup time and space. The bottom surface of the magazine provides a mounting surface for the needle to be inserted substantially perpendicular to a skin surface of a patient. The mounting surface reduces injection variability by providing a large flat contact surface around an insertion site of the skin of the patient, thereby improving injection comfort. In addition, since the needle is never removed from the magazine, the needle is barely seen and thus reduces needle phobia.

The needle magazine also reduces the likelihood of needle reuse in several ways. First, the needle magazine has a seal on a top surface that acts as a visual indicator to indicate to a user whether the needle is used or new. Second, when the needle is withdrawn after use, the needle is in a locked position preventing further movement and reuse. Finally. after the needle is locked, the needle is positioned deeper within a cavity of the magazine to provide the user a visual indication that the needle is used.

The foregoing and/or other aspects of the present invention can be achieved by providing a needle storage container comprising a first compartment including a needle magazine capable of carrying a plurality of unused and used needles. and an adapter configured to engage a medication delivery pen, and a second compartment configured to carry a cleaning agent, wherein the first compartment is sealed from the second compartment.

The foregoing and/or other aspects of the present invention can further be achieved by providing a needle magazine comprising a plurality of cavities each carrying a needle fixed to a needle post, a first seal disposed at a proximal end of each of the plurality of cavities, and a second seal disposed at a distal end of each of the plurality of cavities, wherein in a first position of the needle post, the needle is enclosed between the first and second seals and a distal end of the needle is disposed into the second seal, in a second position of the needle post, the first seal is pierced and the needle pierces the second seal for medication delivery, and in a third position of the needle post, the needle no longer pierces the second seal and is ready for disposal.

The foregoing and/or other aspects of the present invention can also be achieved by providing a pen assembly comprising a medication delivery pen, an adapter configured to be engaged to the medication delivery pen, and a needle magazine having a plurality of cavities that each carry a needle fixed to a needle post, wherein when the adapter is attached to the medication delivery pen and disposed into one of the plurality of cavities, fluid communication is established with the needle in the needle post and the medication delivery pen, and the needle post is moved from a first position to a second position in the cavity so that the needle extends beyond a bottom surface to provide medication delivery.

The foregoing and/or other aspects of the present invention can additionally be achieved by providing a method of using a plurality of needles stored in needle magazine, the method comprising connecting an adapter to a medication delivery pen, engaging the adapter to a needle post carrying the needle to establish fluid communication between the medication delivery pen and the needle, moving the needle post downward from a first position to a second position, administering medication via the needle, moving the needle post upward from the second position to a third position. and sending the needle magazine to a manufacturer or a waste management entity after use of all of the plurality of needles in the magazine.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 is an exemplary embodiment of a right perspective view of a needle storage container including needle magazines and an adapter;

FIG. 2 is a left perspective view of the needle storage container of FIG. 1;

FIG. 3 is atop perspective view of the needle magazine of FIG. 1;

FIG. 4 is a cross-sectional view of a pen assembly including a medication delivery pen, the adapter and the needle magazine of FIG. 3;

FIG. 5 is a cross-sectional view of the pen assembly of FIG. 4 with the medication delivery pen aligned with a cavity of the needle magazine;

FIG. 6 is a cross-sectional view of the pen assembly of FIG. 5 with the adapter connected to the medication delivery pen and piercing a first seal in the needle magazine;

FIG. 7 is a cross-sectional view of the pen assembly of FIG. 6 with the adapter connected to the medication delivery pen and engaged to the needle post in the needle magazine;

FIG. 8 is a cross-sectional view of the pen assembly of FIG. 7 with the adapter connected to the medication delivery pen and moving the needle post from a first position to a second position;

FIG. 9 is a cross-sectional view of the pen assembly of FIG. 8 with a cap removed from the needle magazine to prepare for medication delivery;

FIG. 10 is a front elevation view of the pen assembly of FIG. 9 where the medication delivery pen is in fluid communication with a needle in the needle post and delivering medicament into a skin of a patient;

FIG. 11 is a cross-sectional view of the pen assembly of FIG. 8 with the cap replaced after medication delivery by the selected needle: and

FIG. 12 is a cross-sectional view of the pen assembly of FIG. 11 with the needle post moved from the second position to a third position, and

FIG. 13 is a cross-sectional view of the pen assembly of FIG. 12 with the adapter connected to the medication delivery pen and disengaged from the needle post in the needle magazine.

### DETAILED DESCRIPTION OF EXEMPLARY EMBODIMENTS

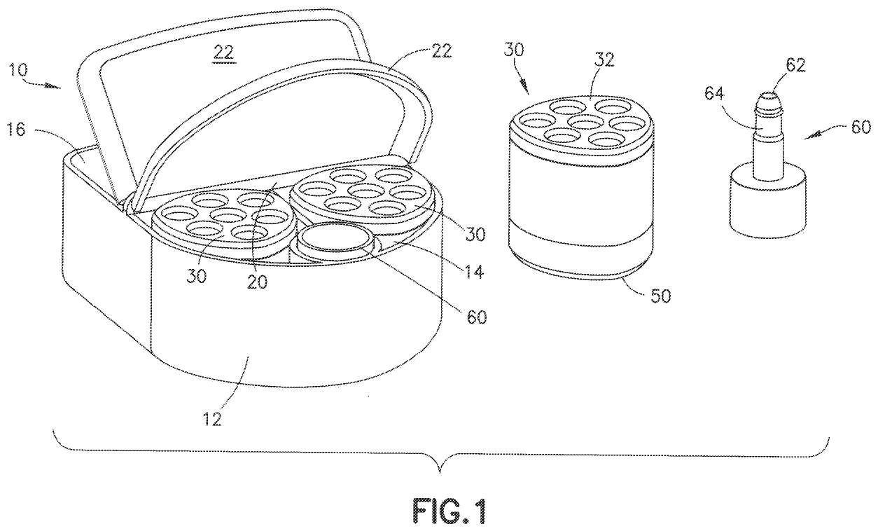

FIG. 1 illustrates a needle storage container 10, according to one embodiment, including a main housing 12 partitioned into a first compartment 14 and a second compartment 16. The first compartment is configured to carry two or more needle magazines 30 and an adapter 60, while the second compartment 16 is configured to carry a cleaning agent 18. A divider wall 20 partitions the first and second compartments 14, 16. Each compartment 14, 16 is covered by a compartment cover 22.

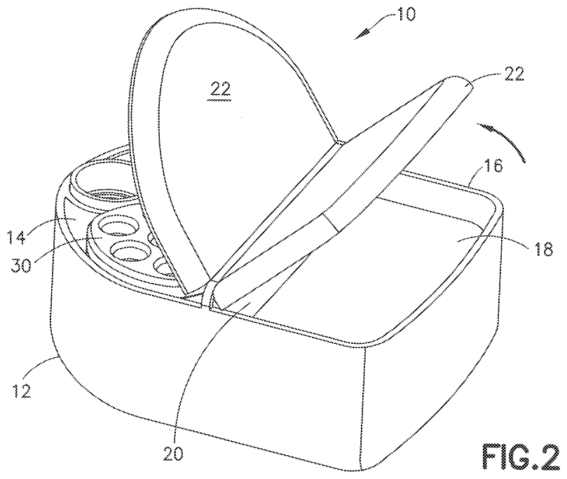

FIGS. 1 and 2 illustrate the main housing 12 being an outer container-like structure enclosing the components described herein. The first compartment 14 is of a substantially semicircular shape and is separated into three sections, although more or less sections are contemplated. Two sections are used to carry each of the needle magazines 30 and the third section is used to carry the adapter 60. Further details regarding the needle magazine 30 and the adapter 60 are described below.

The second compartment 16 is of a substantially rectangular shape and includes the cleaning agent 18 such as an alcohol swab, for example. Other cleaning agents 18 commonly known by those skilled in the art are contemplated herein. The cleaning agent 18 is used to sanitize an adapter septum 62 or a septum 6 of a medication delivery pen 2 prior to or after use.

The divider wall 20 separates the first compartment 14 from the second compartment 16. The divider wall 20 is unitary to the main housing 12 to provide an interface that separates the two compartments 14, 16 and acts as a hermetic, liquid or airtight seal. Alternately, the divider wall 20 can be a feature inserted into the main housing 12 and integrated with seals to provide similar sealing benefits described above.

Finally, each of the compartments 14, 16 includes a compartment cover 22. The compartment covers 22 are each disposed on top surface of one of the compartments 14, 16. The compartment covers 22 are hinged above the divider wall 20 such that the compartment covers 22 open and close in opposing directions. Accordingly, the compartments covers 22 open and close to provide access and restrict access to the contents in each of the compartments 14, 16.

FIG. 1 further illustrates the adapter 60 used to establish fluid communication between the medication delivery pen 2 and one of a plurality of needles 74 in the needle magazine 30. The adapter 60 includes the adapter septum 62, a snap ring member 64, an inner thread 66 and a hollow protruding portion 68. The adapter 60 is a hollow member that is configured to allow fluid to pass through.

The adapter septum 62 is disposed at a distal end and on an internal surface of the hollow adapter 60 to selectively allow or restrict fluid flow. The adapter septum 62 is configured to engage one of the plurality of needles 74 in the needle magazine 30. Specifically, the proximal end of one of the needles 74 is configured to pierce the adapter 62 to establish fluid communication.

The snap ring member 64 is a circumferential protrusion disposed on an external surface of the adapter 60. The snap ring member 64 is also disposed near the distal end of the adapter 60. The plurality of needles 74 is each fixed to a needle post 70 and the needle post 70 is configured to engage the snap ring member 64 when one of the needles 74 pierces the adapter septum 62. This engagement is described below in more detail.

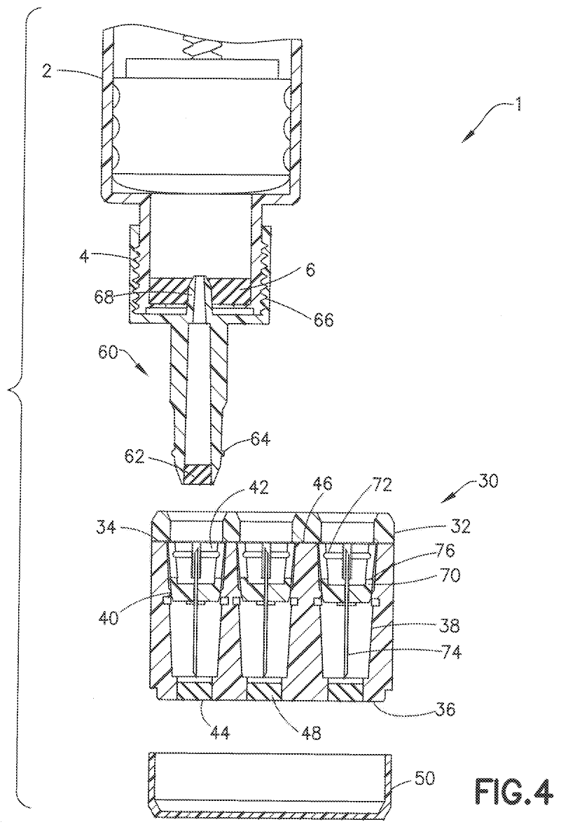

As illustrated in FIG. 4, the inner thread 66 is disposed on a proximal end of the adapter 60 and is configured to engage the medication delivery pen 2. The adapter 60 also includes the hollow protruding portion 68, comprising a spike or a needle, for example, that is disposed within a cavity of the inner thread 66. The hollow protruding portion 68 is configured to pierce the septum 6 of the medication delivery pen 2.

Specifically. the medication delivery pen 2 includes an external thread 4 and the septum 6 as commonly understood by one skilled in the art. The external thread 4 is configured to thread into and engage the inner thread 66 of the adapter 60. The septum 6 is a seal that selectively holds medicament inside a cartridge of the medication delivery pen 2. Fluid communication is established between the medication delivery pen 2 and the adapter 60 when the hollow protruding portion 68 surrounded by the inner thread 66 of the adapter 60 pierces the septum 6 and the inner thread 66 fully engages the external thread 4.

FIG. 4 illustrates a pen assembly 1 including the medication delivery pen 2, and the needle magazine 30 and the adapter 60 removed from the needle storage container 10. FIGS. 1, 3 and 4 illustrate the needle magazine 30 used to retain the plurality of needles 74. The needle magazine 30 includes a retainer plate 32, a top surface 34, a bottom surface 36, a plurality of cavities 38. first and second seals 46, 48 and a cap 50. The needle magazine 30 is a circular member carrying the plurality of needles 74, preferably seven, although more or less is contemplated.

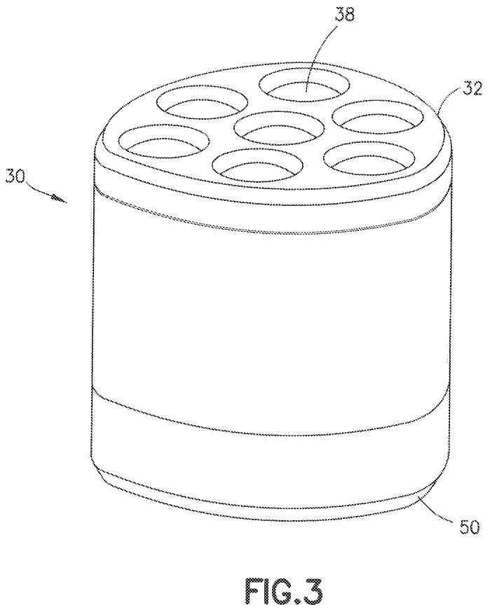

The retainer plate 32 is a thick, circular sheet disposed on a top surface 34 as illustrated in FIGS. 3 and 4. Seven equally spaced holes are disposed through the retainer plate 32 to provide access to the plurality of needles 74. Specifically, the plurality of cavities 38 carry the plurality of needles 74 and are aligned to the holes of the retainer plate 32 to advantageously orient the medication delivery pen 2 attached to the adapter 60 and reduce angled insertion of the adapter 60 into one of the cavities 38.

Each of the plurality of cavities 38 is tapered so that a diameter of one of the cavities 38 at a proximal end 42 is greater than a diameter of the cavity 38 at a distal end 44. The plurality of cavities 38 being tapered advantageously provides increased friction and engagement between the needle post 70 and the adapter 60 when the needle post 70 travels from a first position, to a second position and to a third position. Further description of these positions is set forth below.

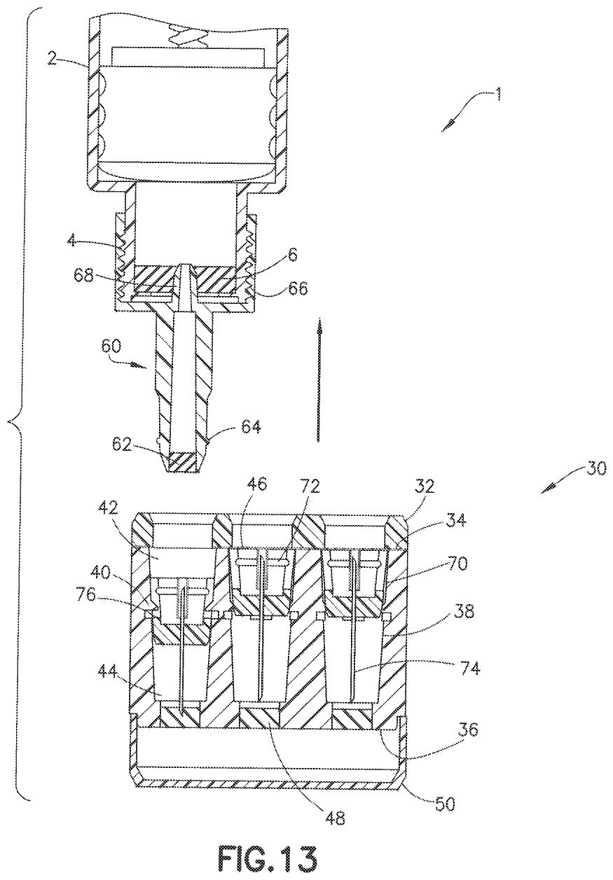

The plurality of cavities 38 includes a protrusion 40, as best illustrated in FIG. 13. The protrusion 40 is a protruded internal triangular surface that is configured to engage a locking member 76 of each of the plurality of needle posts 70. This engagement takes place in the third position of the needle post 70 to lock the needle post 70 to the cavity 38 and prevent further movement.

The needle magazine 30 also includes the plurality of first seals 46 disposed near a proximal end of the cavities 38 and on the top surface 34 aligned with each of the cavities 38. The plurality of first seals 46 is sandwiched between the retainer plate 32 and the top surface 34 and is adhered to the top surface 34. Preferably. the first seals 46 individual enclose each of the plurality of cavities 38. Once one of the first seals 46 is pierced, the first seal 46 is not reusable.

The first seals 46 are composed of any combination of a plastic, polymer, foil or metallic material. The material of the first seals 46 provides a hermetic, liquid or airtight seal for each of the cavities 38 in the magazine 30. Preferably, the first seals 46 are colored in green, orange or any other visibly contrasting color, for example. The colored first seals 46 advantageously provide a visually distinct appearance that the respective cavity 38 has not been accessed.

Alternately, the needle magazine 30 includes a first seal 46 comprising a single sheet that spans the top surface 34 and sandwiched between the top surface 34 and the retainer plate 32. In this configuration, different portions of the first seal 46 corresponding to and aligned with each of the plurality of cavities 38 are pierced when the cavity 38 is accessed and used.

The second seal 48 is disposed in each of the cavities 38 near a distal end and adjacent to the bottom surface 36. The second seal 48 more closely functions as a reusable septum or valve. The use of the seals 46, 48 during the operation of the needle magazine 30 is described below.

Prior to use of the needle magazine 30, each needle 74 and the needle post 70 is in the first position between the first and second seals 46, 48. The distal end of the needle 74 is preferably disposed above the second seal 48 or alternately disposed in the second seal 48. In either case, the distal end of the needle 74 does not extend through the second seal 48 in this position. The presence and color of the first seals 46 advantageously indicate that the respective cavity 38 has not been previously accessed. Subsequently, the user moves the adapter 60 attached to the medication delivery pen 2 to pierce the one of the first seals 46 in one of the cavities 38 and access the needle 74 and needle post 70.

Next, the adapter 60 attached to the medication delivery pen 2 engages the needle post 70 and moves the needle post 70 downward from the first position to the second position. In the second position, the distal end of the needle 74 pierces the second seal 48, extends through the second seal 48 and beyond the bottom surface 36 of the magazine 30. The needle 74 is now ready for medication delivery.

After medication delivery, the adapter 60 attached to the medication delivery pen 2 moves the needle post 70 upward from the second position to the third position. In this position, the distal end of the needle 74 is disposed in the second seal 48 (see FIG. 13) and the needle post 70 is locked to the cavity 38 via the protrusion 40 of the cavity 38 and the locking member 76 of the needle post 70. The distal end of the needle 74 does not extend through the second seal 48 in this position. The pierced first seal 46 is no longer usable and the user can now visibly see that the needle 74 in the cavity 38 was used.

FIGS. 3 and 4 also illustrate that the needle magazine 30 includes a removable cap 50 that covers the bottom surface 36 and the distal end 44 of the plurality of cavities 38. The cap 50 is a transparent member that is engaged to the bottom surface 36 to protect the needle 74 from bending or misuse such as inadvertent sticking. The cap 50 also advantageously protects the bottom surface 36 from damage since the bottom surface 36 is the mounting surface used when medicament is delivery into the skin of the patient. When the needle 74 is ready for medication delivery and the needle 74 extends beyond the bottom surface 36, the cap is removed so that the needle 74 is exposed.

The bottom surface 36 advantageously provides an increased mounting surface to reduce localized pressure at the insertion site by spreading the pressure or injection force evenly across the surface area of the mounting surface. The mounting surface also advantageously improves accurate and consistent needle depth into the skin surface by automatically stretching and flattening the skin prior to injection of the needle 74. Specifically, if the needle 74 is inserted beyond a subcutaneous region of the skin, a muscle of the patient may be undesirably accessed. If the needle 74 is inserted and placed in a dermis or epidermis region of the skin instead of the subcutaneous region, the patient may not experience proper medication effectiveness. Finally, the mounting surface advantageously provides a flat surface so that the needle 74 can be inserted accurately at substantially a 90° angle with respect to the surface of the skin. Such a mounting surfaces improves injection repeatability and reduces injection variability.

Each of the plurality of cavities 38 includes the needle post 70 fixed to the respective needle 74. Each needle post 70 includes a snap ring groove 72 and a locking member 76. The snap ring groove 72 is configured to engage the snap ring member 64 of the adapter 60. Specifically, when the adapter 60 is connected to the medication delivery pen 2 and enters into one of the plurality of cavities 38, the snap ring groove 72 of the needle post 70 mates with the snap ring member 64 of the adapter 60. This engagement allows the adapter 60 connected to the medication delivery pen 2 to move the needle post 70 from the first position, to the second position and to the third position.

The locking member 76 of the needle post 70 is a plurality of linear slots circumferentially disposed on an exterior surface of the needle post 70. One of the plurality of linear slots of the locking member 76 is configured to engage the protrusion 40 in each of the cavities 38. Alternately, the protrusion 40 can engage a top surface of the needle post 70 to lock the needle post 70 in the cavity 38. The needle post 70 moves between three positions while interacting with the protrusion 40. These positions are briefly described below.

In the first position, the needle post 70 is disposed between the first and second seals 46, 48, the distal end of the needle 74 is preferably disposed in the second seal 48, and the needle 74 is ready for use. In this position, an outer surface of the needle post 70 compresses the protrusion 40.

In the second position, the needle post 70 is moved downward from the first position by the adapter 60 connected to the medication delivery pen 2. In this position, the needle 74 pierces and extends through the second seal 48 and is ready for medication delivery. In addition, the needle post 70 is moved past the protrusion 40 of the cavity 38 where the protrusion 40 is disposed above the needle post 70.

After the needle 74 is used, the needle post 70 is moved to the third position. In the third position, the needle post 70 moves upward from the second position and the locking member 76 engages the protrusion 40 of the cavity 38. In this position, the needle post 70 is locked in the cavity 38 and unable to move to the first or second positions, and also cannot be removed from the cavity 38. In addition, the distal end of the needle 74 is disposed in the second seal 48 and no longer extends through the second seal 48. Finally, the third position of the needle post 70 is between the first position and the second position. Accordingly, the needle post 70 is disposed in a position recessed from the top surface 34 compared to the first position to advantageously indicate to the user that the needle 74 is used.

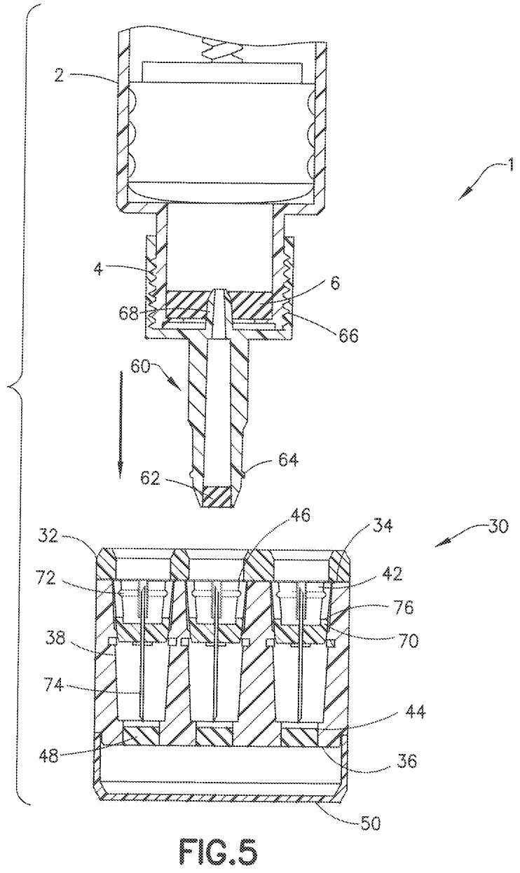

The detailed operation of the pen assembly 1 is described as follows. FIG. 5 illustrates the pen assembly 1 where one of the needle magazines 30 and the adapter 60 are removed from the first compartment 14 of the needle storage container 10. As illustrated in FIGS. 1 and 2, the user may optionally clean the bottom surface 36 of the needle magazine 30 and the adapter septum 62 by using the cleaning agent 18 disposed in the second compartment 16 of the needle storage container 10.

The adapter 60 is also attached to the medication delivery pen 2. Specifically, the inner threads 66 of the adapter 60 engage the external thread 4 of the medication delivery pen 2. When the inner thread 66 and the external thread 4 are fully engaged, the hollow protruding portion 68 of the adapter 60 pierces the septum 6 of the medication delivery pen 2 to establish fluid communication. Subsequently, the user aligns the medication delivery pen 2 to one of the plurality of cavities 38 in the needle magazine 30. The presence and color of the first seals 46 advantageously indicate that the respective cavity 38 has not been previously accessed. The pen assembly 1 is now ready for operation.

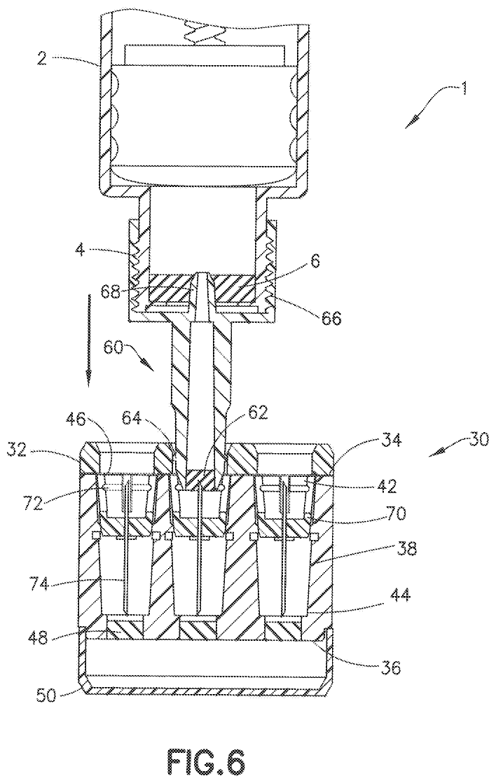

FIG. 6 illustrates the user pushing the adapter 60 connected to the medication delivery pen 2 into one of the plurality of cavities 38 in the needle magazine 30. First, the adapter 60 enters one of the holes aligned with one of the cavities 38 in the retainer plate 32. The retainer plate 32 advantageously orients the medication delivery pen 2 and reduces angled insertion of the adapter 60 into one of the cavities 38. Next, the adapter 60 pierces the first seal 46 to access the needle 74 in one of the cavities 38. The first seal 46 is a single use seal for hermetic, liquid or airtight sealing of each of the plurality of cavities 38.

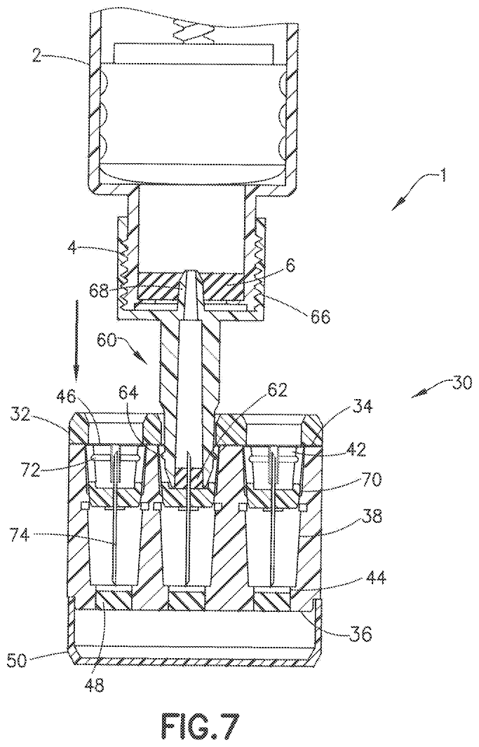

FIG. 7 illustrates the adapter 60 connected to the medication delivery pen 2 and engaged to one of the needle posts 70 disposed in the first position, specifically, the snap ring member 64 of the adapter 60 is engaged with the snap ring groove 72 of the needle post 70. At the same time, the proximal end of the needle 74 pierces the adapter septum 62 of the adapter 60. Thus, the distal end of the needle 74 is in fluid communication with the medication delivery pen 2. In the first position of the needle post 70, the distal end of the needle 74 is preferably disposed above the second seal 48 or alternately disposed in the second seal 48. In either case, the distal end of the needle 74 does not extend through the second seal 48 in this position. Also, in the first position, the outer surface of the needle post 70 compresses the protrusion 40 of the cavity 38.

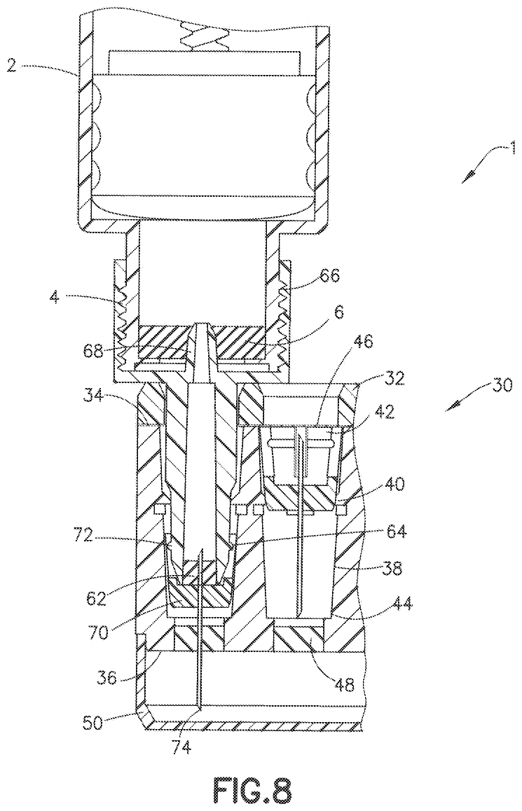

FIG. 8 illustrates the adapter 60 connected to the medication delivery pen 2 pushing the needle post 70 from the first position to the second position. The second position is reached when a distal surface of the medication delivery pen 2 contacts a top surface of the retainer plate 32. In the second position, the needle post 70 moves past the protrusion 40 in the cavity 38. The distal end of the needle 74 extends through the second seal 48.

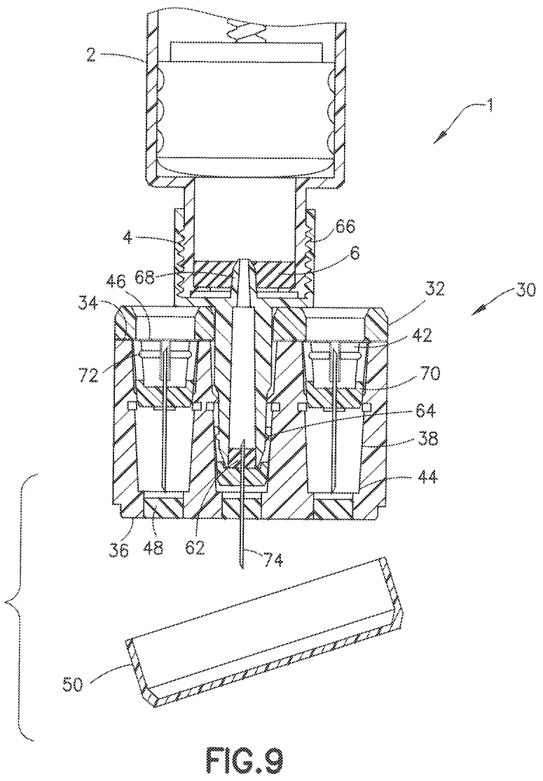

The cavity 38 is narrower as the needle post 70 moves from the first position to the second position. The tapered cavity 38 advantageously strengthens the engagement between the snap ring member 64 of the adapter 60 and the snap ring groove 72 of the needle post 70. This strengthened engagement reduces the likelihood of inadvertent disengagement of the adapter 60 to the needle post 70 when the needle post 70 is moving. FIG. 9 illustrates the cap 50 removed from the bottom surface 36 of the needle magazine 30. The needle 74 is now ready for medication delivery.

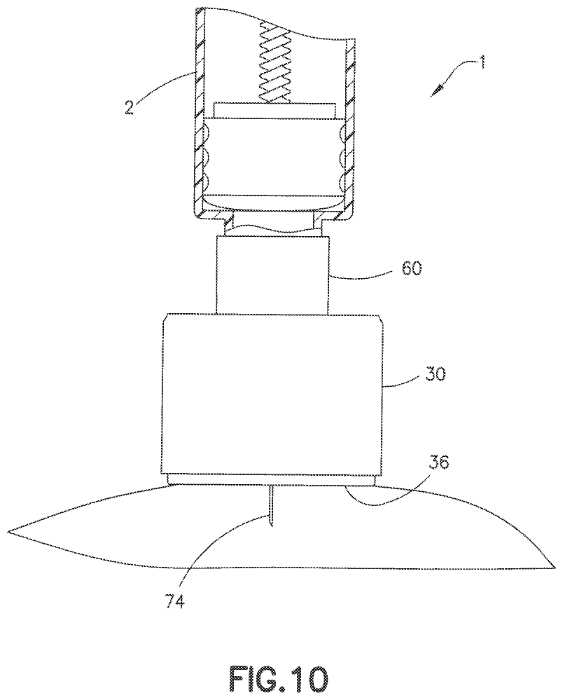

FIG. 10 illustrates the adapter 60 connected to the medication delivery pen 2 and engaged to the needle magazine 30 in the second position of the needle post 70 where the needle 74 is inserted into the skin of the patient for medication delivery. Medication is advantageously being delivered while the needle 74 is in the needle magazine 30. This configuration minimizes the sight of the needle 74 to reduce anxiety for users and/or patients having needle phobia.

In addition, the bottom surface 36 of the needle magazine 30 contacts the skin of the patient during needle insertion and medication delivery. The bottom surface 36 of the magazine advantageously provides an increased and flat mounting surface for the needle 74 to be inserted substantially perpendicular to the skin surface of the patient. The mounting surface further reduces localized pressure at the insertion site and injection pain by providing a large flat contact surface around the insertion site of the skin of the patient that spreads the pressure or injection force evenly across the mounting surface. The mounting surface also advantageously improves accurate and consistent needle depth into the skin surface by automatically stretching and flattening the skin prior to injection of the needle 74.

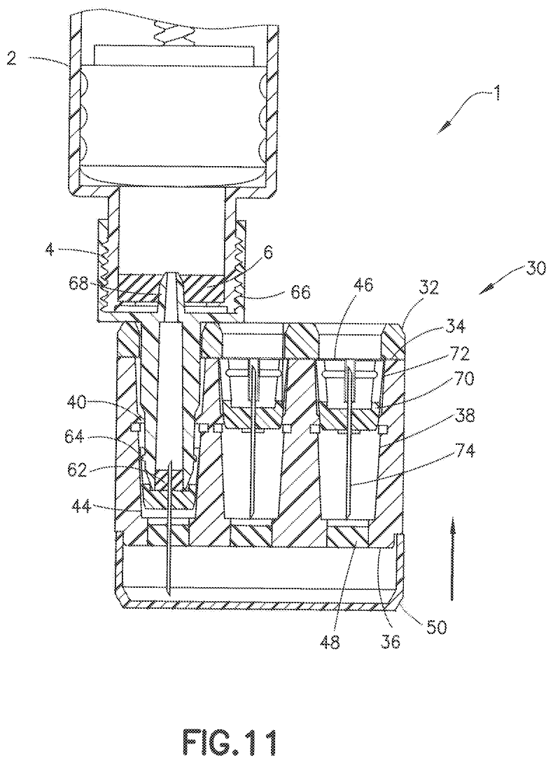

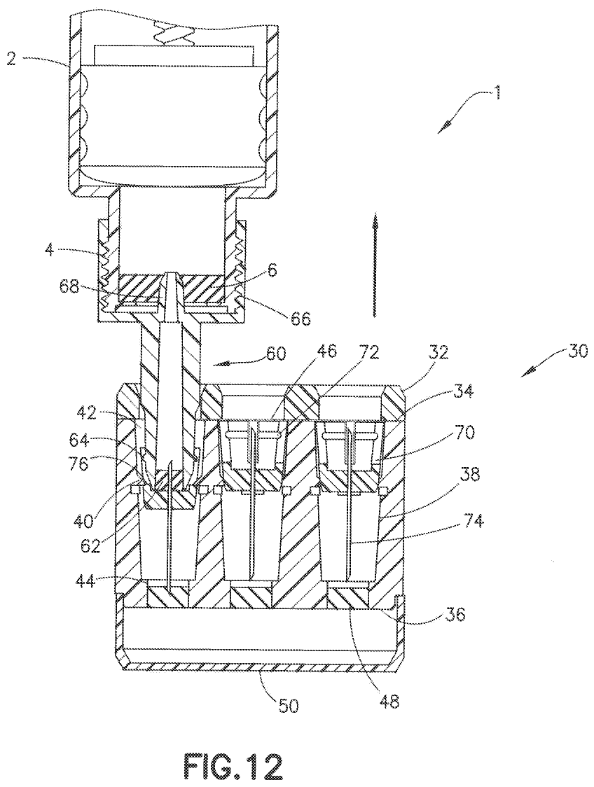

After medication delivery, FIG. 11 illustrates the cap 50 disposed over the bottom surface 36 of the needle magazine 30 to shield the distal end of the needle 74. FIG. 12 illustrates the needle post 70 moved upward from the second position to the third position. In the third position, the protrusion 40 of the cavity 38 engages the locking member 76 of the needle post 70 to lock the needle post 70 from any further movement in the cavity 38. The tapered cavity 38 advantageously reduces the likelihood that the adapter 60 will prematurely disengage from the needle post 70 prior to the needle post 70 locking into the cavity 38 in the third position.

The third position also illustrates that the needle 74 is retracted into the cavity 38 and the distal end of the needle 74 is disposed in the second seal 48. The distal end of the needle 74 does not extend through the second seal 48 in this position. The third position of the needle post 70 is between the first position and the second position. Accordingly, the needle post 70 in the third position is advantageously recessed from the top surface 34 so that the user can visually see that the needle 74 has been used.

FIG. 13 illustrates the adapter 60 connected to the medication delivery pen 2 being disengaged from the needle post 70 and removed from the cavity 38. Accordingly, the needle 74 is no longer in fluid communication with the medication delivery pen 2. The user can then use another needle 74 in another one of the plurality of cavities 38 and repeat the operational steps described above.

After all the needles 74 have been used and placed in the third position in the cavity 38 of the needle magazine 30, the user can advantageously send the needle magazine 30, via mail, for example, to a manufacturer or a waste management entity for safe and sterile disposal. Optionally. the manufacturer can advantageously recycle and reuse various components of the needle magazine 30.

For example, the manufacturer can sterilize and reuse some or all of the cavities 38 in the needle magazine 30. Specifically, the manufacturer can replace the used needle 74 with the unused needle 74 in one or more cavities 38 and seal the respective cavity 38 with seals 46, 48. The needle magazine 30 can then be shipped and ultimately sold for reuse.

In another example, the needle storage container 10 can be sent back to the manufacturer. The manufacturer can replace the cleaning agent 18 in the second compartment 16. In addition, all the needles 74 in the magazines 30 and the adapter 60 can be replaced. The needle storage container 10 can then be shipped and ultimately sold for reuse.

The storage and use of the needles 74, as well as the disposal of the needles 74 in the same needle magazine 30 provides many advantages and benefits. There is no need for separate packages for storage and disposal of needles 74. This is because the needle magazine 30 advantageously synchronizes needle 74 use and disposal. This versatile arrangement provides better usage, optimizes workflow, minimizes setup time and optimizes space.

The foregoing detailed description of the certain exemplary embodiments has been provided for explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. In addition, any of the embodiments, features and/or elements disclosed herein may be combined with one another to form various additional combinations not specifically disclosed, as long as the embodiments, features and/or elements being combined do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges around and including the given value, for example, general tolerances associated with manufacturing, assembly, and use of the described embodiments.

1. A needle storage container (10) comprising:
a first compartment (14) including
a needle magazine (30) capable of carrying a plurality of unused and used needles (74); and
an adapter (60) configured to engage a medication delivery pen (2); and
a second compartment (16) configured to carry a cleaning agent (18); wherein
the first compartment (14) is sealed from the second compartment (16).

2. The needle storage container (10) of claim 1, further comprising:
a main housing (12) including the first and second compartments (14, 16); and
a wall (20) separating the first compartment (14) from the second compartment (16).

3. The needle storage container (10) of claim 2, further comprising compartment covers (22) enclosing each of the first and second compartments (14, 16).

4. The needle storage container (10) of claim 1, wherein a proximal end of the adapter (60) includes a septum (62).

5. The needle storage container (10) of claim 1, wherein
the adapter (60) includes a snap ring member (64) that is configured to engage a needle post (70) carrying the needle (74) in the needle magazine (30); and
the needle (74) remains in the needle magazine (30) during medication delivery.

6. A needle magazine (30) comprising:
a plurality of cavities (38) each carrying a needle (74) fixed to a needle post (70);
a first seal (46) disposed at a proximal end of each of the plurality of cavities (30); and
a second seal (48) disposed at a distal end of each of the plurality of cavities (30); wherein
in a first position of the needle post (70), the needle (74) is enclosed between the first and second seals (46, 48);
in a second position of the needle post (70), the first seal (46) is pierced and the needle (74) pierces and extends through the second seal (48) for medication delivery (2); and
in a third position of the needle post (70), the needle (74) no longer extends through the second seal (48) and is ready for disposal.

7. The needle magazine (30) of claim 6, further comprising:
a retainer plate (32) disposed on a top surface (34): wherein
the first seals (46) are disposed between the retainer plate (32) and the top surface (34).

8. The needle magazine (30) of claim 6, wherein the plurality of cavities (38) is tapered so that a distal end of each cavity (38) is smaller in diameter than a proximal end of each cavity (38).

9. The needle magazine (30) of claim 6, further comprising a cap (50) enclosing a bottom surface (36).

10. The needle magazine (30) of claim 6, wherein in the second position, the needle post (70) moves downward from the first position.

11. The needle magazine (30) of claim 6, wherein in the third position, the needle post (70) moves upward from the second position.

12. The needle magazine (30) of claim 6, wherein the third position is between the first position and the second position.

13. A pen assembly (1) comprising:
the needle magazine (30) of claim 6;
a medication delivery pen (2); and
an adapter (60) configured to engage the medication delivery pen (2) at a proximal end and configured to engage the needle post (70) at a distal end.

14. The pen assembly (1) of claim 13, wherein the adapter (60) includes a snap ring member (64) that is configured to engage a snap ring groove (72) of the needle post (70).

15. The pen assembly (1) of claim 13, wherein the adapter (60) includes a protrusion that is configured to pierce a septum (6) of the medication delivery pen (2) to establish fluid communication.

16. The pen assembly (1) of claim 13, wherein a distal end of the adapter (60) includes a septum (62) that is configured to be pierced by the needle (74) in the needle post (70).

17. The pen assembly (1) of claim 13, wherein the adapter (60) pushes the needle post (74) from the first position to the second position causing the adapter (60) to pierce the first seal (46) and the needle (74) to pierce and extend through the second seal (48).

18. The pen assembly (1) of claim 13, wherein in the second position of the needle post (74),
the medication delivery pen (2) is connected to the adapter (60) and the adapter (60) is engaged to the needle post (70);
a distal end of the needle (74) is in fluid communication with the medication delivery pen (2) and is ready for medication delivery; and
a bottom surface (36) is configured to contact a skin surface to provide a mounting surface for injection.

19. The pen assembly (1) of claim 13, wherein the adapter (60) pulls the needle post (70) upwardly from the second position to the third position to retract the needle (74) for disposal.

20. The pen assembly (1) of claim 13, wherein in the third position of the needle post (70),
the needle (74) is not in fluid communication with the medication delivery pen (2); and
the needle post (70) includes a locking member (76) that engages a protrusion (40) in the cavity (38) to lock the needle post (70) in the third position.

21. A pen assembly (1) comprising:
a medication delivery pen (2);
an adapter (60) configured to be engaged to the medication delivery pen (2); and
a needle magazine (30) having a plurality of cavities (38) that each carry a needle (74) fixed to a needle post (70); wherein
when the adapter (60) is attached to the medication delivery pen (2) and disposed in one of the plurality of cavities (38), fluid communication is established with the needle (74) in the needle post (70) and the medication delivery pen (2); and
the needle post (70) is moved from a first position to a second position in the cavity (38) so that the needle (74) extends beyond a bottom surface (36) to provide medication delivery.

22. A method of using a plurality of needles (74) stored in a needle magazine (30), the method comprising:
connecting an adapter (60) to a medication delivery pen (2);
engaging the adapter (60) to a needle post (70) carrying the needle (74) to establish fluid communication between the medication delivery pen (2) and the needle (74);
moving the needle post (70) downward from a first position to a second position;
administering medication via the needle (74);
moving the needle post (70) upward from the second position to a third position; and
sending the needle magazine (30) to a manufacturer or a waste management entity after use of all of the plurality of needles (74) in the needle magazine (30).

23. The method of claim 22, further comprising piercing a first seal (46) of one of the cavities (38) of the needle magazine (30) prior to the step of engaging the adapter (60) to the needle post (70).

24. The method of claim 22, further comprising piercing a second seal (48) of one of the cavities (38) of the magazine (30) prior to the step of administering medication.

25. The method of claim 22, further comprising disposing a bottom surface (36) of the needle magazine (30) on an insertion site prior to the step of administering medication.

26. The method of claim 22, further comprising placing a cap (50) on a bottom surface (36) prior to the step of sending the needle magazine to a manufacturer.
